(19) United States
(12) Patent Application Publication
(10) Pub. No.: US 2017/0079832 A1
(43) Pub. Date: Mar. 23, 2017

(54) FEMALE URINARY BARRIER SYSTEM

(71) Applicant: Heng Henry Khun, Calgary (CA)

(72) Inventor: Heng Henry Khun, Calgary (CA)

(21) Appl. No.: 14/859,607

(22) Filed: Sep. 21, 2015

(51) Int. Cl. A61F 6/06; A61F 2/00

(52) U.S. Cl. CPC A61F 6/06; A61F 2/0009; A61F 2220/0008; A61F 2230/0019; A61F 2250/009

(57) ABSTRACT

A female urinary barrier system product that includes an exterior vaginal attachment pad for providing a female-user with means to prevent the spread of infectious diseases into their vaginal opening and urinary tract. The product provides a female-user a hand depressible attachment pad useful for enclosing/covering the vagina prior to a bowel movement or just prior to wiping. The product embodies a barrier that protects a female-user's urethra from fecal matter and other external harmful pathogenic bacteria, viruses, and fungi entering therein. Another version of the present invention is an attachable pad applicable to an undergarment of a female-user for purposes of prevention of bacterial inoculation from exterior sources.

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of infection prevention devices and more specifically relates to a female vaginal infectious disease contamination prevention barrier especially for use by females to prevent urinary tract infections.

2. Description of Related Art

Personal hygiene has become a subject of much importance due to the desire to prevent the spread of disease and infectious agents between people. One particular type of infection most commonly effecting females is a urinary tract infection (UTI). A UTI from a lower urinary infection includes painful urination and either frequent urination or urge to urinate as symptoms. The main infecting agent causing a UTI are Escherichia coli (E-coli), and secondarily other uncommon agents may be bacteria, viruses, or fungi.

Since E-coli is prevalent in fecal matter, the spread of fecal matter to the urethra is often the cause of a UTI. The other frequent risk factors for a UTI include sexual activity, female sex, and having a family history of UTI. Personal hygiene practices and anatomical considerations also factor in reoccurring UTIs. With improper wiping after a bowel movement the spread of fecal matter into the vagina, and eventually to the urethra may occur. Also, shorter urethras and shorter distances between the urethra and the anus can put a female at a disadvantage for contracting a UTI.

High occurrences of UTIs have especially been found in women, with half of women contracting at least one infection at some point in their lives. Fortunately, most cases are easily treated with short courses of antibiotics. Unfortunately, today many doctors have been found to over-prescribe antibiotics, and the resistance of bacteria to many of the antibiotics prescribed today is increasing. This is not a desirable solution.

More complicated cases of UTIs are difficult to treat and require more attentive follow ups by the person affected by seeking more evaluation, and treatment. In the United States, seven million office visits alone occur every year due to the infection. This may be both a financial and time-consuming burden. UTIs also most often occur between the ages 16 and 35 years of age and reoccur with half the people contracting a second infection within a year. In the United States the cost of treatment for UTI's is estimated at 1.6 billion US dollars every year. For those females incurring lost time and wages due to reoccurring UTIs, a more manageable system for preventing the infection is needed.

Several attempts have been made to solve the above-mentioned problems such as those found in U.S. Pat. No. 8,889,655 to Allison London Brown; Canadian Patent No. 1,168,975 to Sven-Olov Odelhog; U.S. Pat. No. 4,372,309 to George B. Fowler; U.S. Publication No. 2010/0021512 to Stanley Anon; US Publication No. 2012/0053546 to Michelle Fogg; Canadian Patent No. 2,557,077 to Yoshitaka Mishima; International Patent No. 2,839,816 to Hidetada Sasaki; U.S. Pat. No. 6,699,228 to Harry Joseph Chmielewski; U.S. Pat. No. 7,204,830 to Yoshitaka Mishima; U.S. Pat. No. 7,666,173 to Yoshitaka Mishima; US Publication No. 2010/0305542 to Gordon A. Stemple; and US Publication No. 2013/0338620 to Jane Speak. This art is representative of personal apparel designed to prevent spread of infections via feces, urine, and other contaminants. However, none of the above inventions and patents, taken either singly or in combination, is seen to describe the invention as claimed.

Preferably, a female urinary barrier system should provide a safe and reliable means to help prevent UTIs and, yet would operate reliably and be manufactured at a modest expense. Thus, a need exists for a reliable female urinary barrier system to avoid the above-mentioned problems associated with UTIs.

### BRIEF SUMMARY OF THE INVENTION

In view of the foregoing disadvantages inherent in the known infection prevention device art, the present invention provides a novel female urinary barrier system. The general purpose of the present invention, which will be described subsequently in greater detail is to provide a female urinary bather system comprising an infection prevention bather which may be applied to a pubic region of a female-user for preventing infectious matter from entering a vaginal area of the female-user.

The present invention may be useful for preventing urinary tract infections in women by use of an adhesive pad placed over the genital surface before a bowel movement, (during a bowel movement, and after a bowel movement) and removed and disposed of after (completion of the wiping process). The product prevents the bacteria/fecal matter from infecting the female urinary tract during a bowel movement. Toilet paper and other cleaning products can transfer bacteria/fecal matter from the anus to the urethra during the wiping process potentially causing Urinary Tract Infection.

A female urinary barrier system is disclosed herein, in a preferred embodiment, essentially comprising a body and an attacher (attaching means). The body may comprise a top surface, a bottom surface, a first side, a second side, a third side, a fourth side, and a thickness. The female urinary barrier system may comprise the body and the attacher in functional combination for use. The parameters of the body of the female urinary barrier system are defined by the top surface, the bottom surface, the first side, the second side, the third side, the fourth side, and the thickness.

The thickness of the body of the female urinary barrier system may comprise at least one united layer. The top surface of the body of the female urinary barrier system may comprise a material useful for preventing penetration through the body of the female urinary barrier system of fecal matter and other contaminants that are harmful to a female urethra.

The attacher of the female urinary barrier system may comprise at least one adhesive, and the bottom surface of the body of the female urinary barrier system may comprise the adhesive such that the body is able to be applied to an undergarment of a female-user for purposes of prevention of bacterial inoculation from exterior sources in alternate embodiments.

A method of using a female urinary barrier system may comprise the steps of: opening; removing the adhesive protector from the pad; applying; depressing; complete the wiping process; removing; and disposing of the present invention.

The present invention holds significant improvements and serves as a female urinary bather system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, female urinary barrier system, constructed and operative according to the teachings of the present invention.

FIG. 1 shows a perspective view illustrating a relationship between a pubic area of a vagina (vaginal opening and urethra opening) of the female-user to her anus according to an embodiment of the present invention.

FIG. 2 is a perspective view illustrating the female urinary barrier system (pad applied to genital surface) during an ‘in-use’ condition according to an embodiment of the present invention.

FIG. 3 is a perspective view illustrating the urethra opening of the female-user and her anus (pad not applied to genital surface) and visually demonstrating how the bacterial pathogens are apt to infect the female (via splashing) according to an embodiment of the present invention.

FIG. 4 is a perspective view illustrating the female urinary barrier system (pad applied to genital surface) during an ‘in-use’ condition whereby pad forms a barrier to bacterial pathogens according to an embodiment of the present invention.

FIG. 5 is a perspective view illustrating the urethra opening of the female-user and her anus (pad not applied to genital surface) and visually demonstrating how the bacterial pathogens are apt to infect the female (via wiping) according to an embodiment of the present invention.

FIG. 6 is a perspective view illustrating the female urinary barrier system (pad applied to genital surface) during an ‘in-use’ condition whereby pad forms a barrier to bacterial pathogens according to an embodiment of the present invention of FIG. 5.

FIG. 7 is a perspective view illustrating ‘after removal’ of the pad according to an embodiment of the present invention.

FIG. 8 is a perspective view illustrating the pad (as applied) as used with children and young adults according to an embodiment of the present invention.

FIG. 9 is a perspective view illustrating an ultra pad (as applied) as used with children and young adults according to an embodiment of the present invention.

FIG. 10 shows perspective views illustrating a front side with a smooth surface and a back side with a sticky surface, respectively, of the pad according to an embodiment of the present invention.

FIG. 11 shows perspective views illustrating a front side with a sticky surface and a back side with a soft, absorbent surface, respectively, of the ultra pad according to an embodiment of the present invention of FIG. 9.

FIG. 12 shows perspective views illustrating large, medium and small embodiments, respectively, of the pad according to an embodiment of the present invention.

The various embodiments of the present invention will hereinafter be described in conjunction with the appended drawings, wherein like designations denote like elements.

### DETAILED DESCRIPTION

As discussed above, embodiments of the present invention relate to a female urinary barrier system and more particularly to a safe and convenient way to protect the vaginal opening of a female from exterior pathogens as used to improve the cleanliness after a bowel movement and extend such protection to everyday common uses.

Generally speaking, embodiments of the present invention relate to a female pathogen protection device, and more particularly to female urinary barrier system. The female urinary barrier system may be designed to provide a female-user with an exterior vaginal attachment pad to prevent the spread of infectious diseases into their urinary tract.

The conventional ‘cleaning’ process after a bowel movement may lead to urinary tract infections in females. The present invention may prevent contamination via toilet paper and other cleaning products that may transfer bacteria and fecal matter from the anus to the (vagina and) urethra during the wiping process. The present invention in a preferred embodiment may prevent urinary tract infections in women by use of an adhesive pad placed over the genital surface before a bowel movement (just prior to wiping over 90% of the time) and then removed and disposed of there-after. The body of the present invention may be designed for a one time use only.

The present invention may be designed to be used easily and safely by a multitude of users, such as designs fit for children, designs fit for adults, and designs fit for a multitude of different anatomical sizes of female in the general population. The use of the present invention is designed to be user-friendly such that a large percentage of the population may feasibly use the device without need of assistance. The application of the present invention by a female-user may be feasible for the visually impaired and many other persons with disabilities.

One embodiment of present invention may function as a hand depressible attachment pad useful for enclosing the vagina prior to a bowel movement (or after a bowel movement prior to wiping). An alternate embodiment of the present invention comprises an attachable pad applicable to an undergarment of a female-user (covered only the genital area). The alternate embodiment may be designed for everyday use and is appliable to a multitude of different undergarment materials. The alternate embodiment may comprise compositions for purposes of prevention of bacterial inoculation from exterior sources and prevention of other pathogens a female-user may encounter in their daily lives. The alternate embodiment may be designed for such uses as while swimming, running, hiking, and other environmental situations where pathogens may proliferate.

The body of the female urinary barrier system comprises a first side, a second side, a third side, a fourth side, and a thickness that embody a rectangular prism form. The height of the thickness of the body is large enough to completely prevent the transverse penetration of fecal materials there-through. The thickness of the body of the female urinary barrier system comprises 0.1-10 percent cotton, paper, and other materials in preferred embodiments. The aforementioned cotton may provide a matrix within the thickness of the body of the present invention in order to deter the movement of fecal materials through the body of the present invention. The aforementioned paper of the thickness of the body of the present invention may be water soluble, thus providing an environmentally friendly composition.

The rigidity of the body of the present invention may be manufactured such that the present invention may maintain a relatively rectangular prism form in order to provide the desired surface area for easy application by a female-user. The present invention may be flexible enough to conform to the anatomy of a multitude of different female-users, during application by a female-user.

An attacher of the female urinary bather system may be provided on to the bottom surface of the body of the female urinary barrier system for attachment to the periphery of the female-user vagina in preferred embodiments. The attacher may comprise an amount of adhesive applied to the bottom surface of the body of the female urinary barrier system such that the pad stays attached to the female-user after application.

A female-user may wipe after a bowel movement, the current invention may then be easily removed without causing added discomfort. The attacher of the female urinary barrier system may comprise glue comprising 1-10 percent medical grade silicone in certain embodiments. Adhesives and glues may vary by percentage of the medical grade silicon.

The attacher of the female urinary barrier system may comprise a bio-adhesive applied to an entire area of the bottom surface of the body of the female urinary barrier system for purposes of creating a seal around a female-user's vaginal opening. Alternatively, the attacher of the female urinary barrier system may comprise a bio-adhesive applied to an outer periphery of the bottom surface of the body of the female urinary barrier system for purposes of creating a seal around said female-user's vaginal opening.

Furthermore, the aforementioned bio-adhesive may be pressure sensitive such that a female-user may properly apply pressure to the present invention when in place by use of one or two hands. The present invention may be attached to the female-users skin surrounding the vagina and alternatively to other hair and tissue local to the surrounding area of the vagina.

The female urinary barrier system, when properly applied by a female-user, may prevent the invasion of Genus's Enterococcus, Escherichia, Klebsiella, Pseudomonas, Candida, Chlamydia, and Mycoplasma (and other microorganisms or spores contained in the feces) from cross contaminating the vagina and urethra from the anus in order to prevent urinary tract infections.

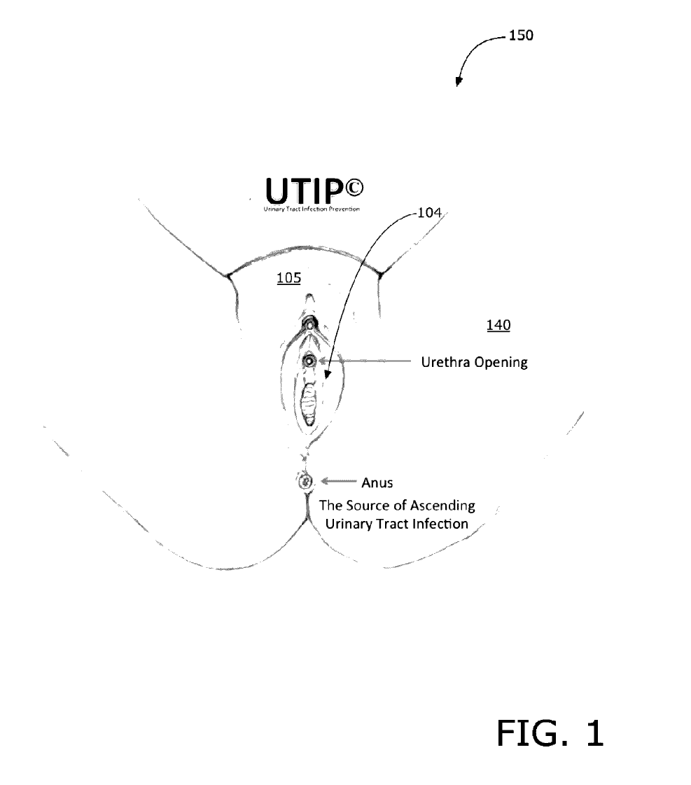

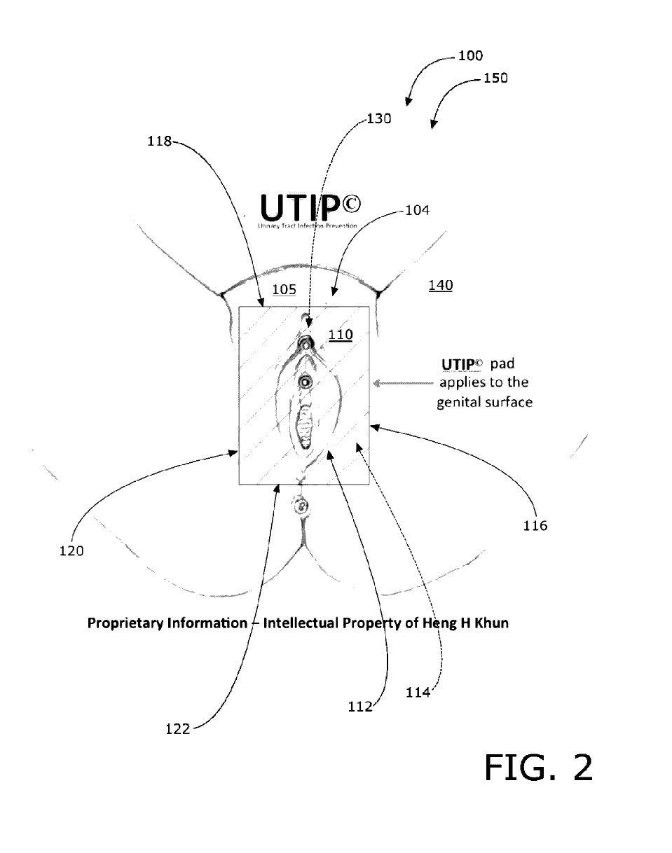

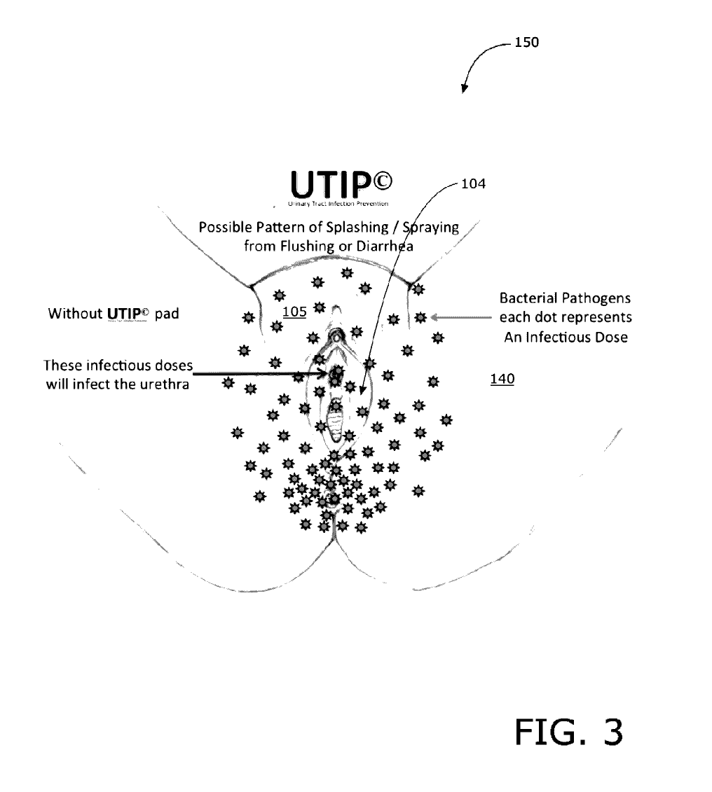

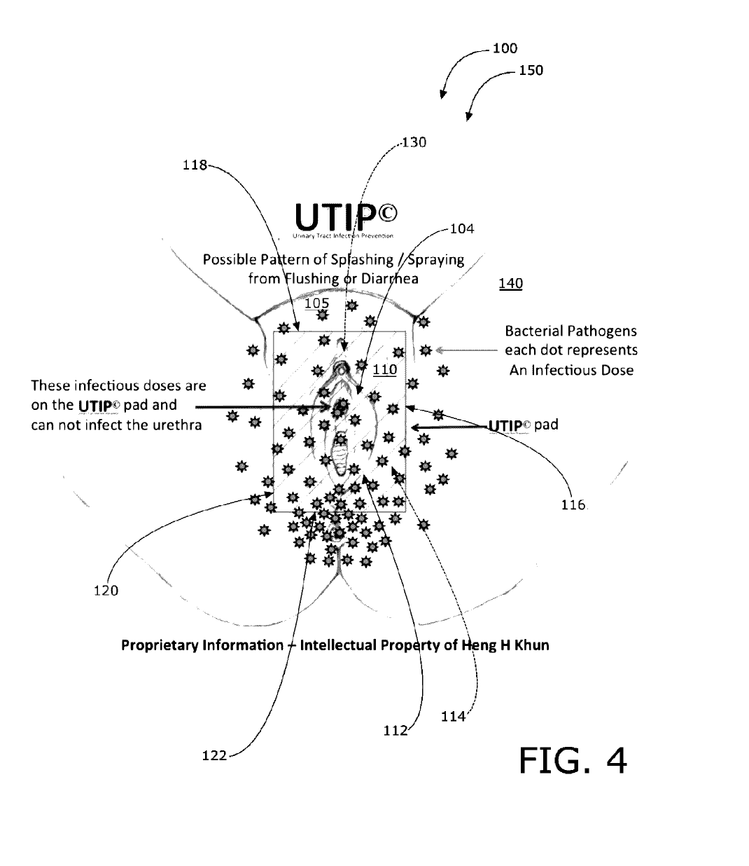

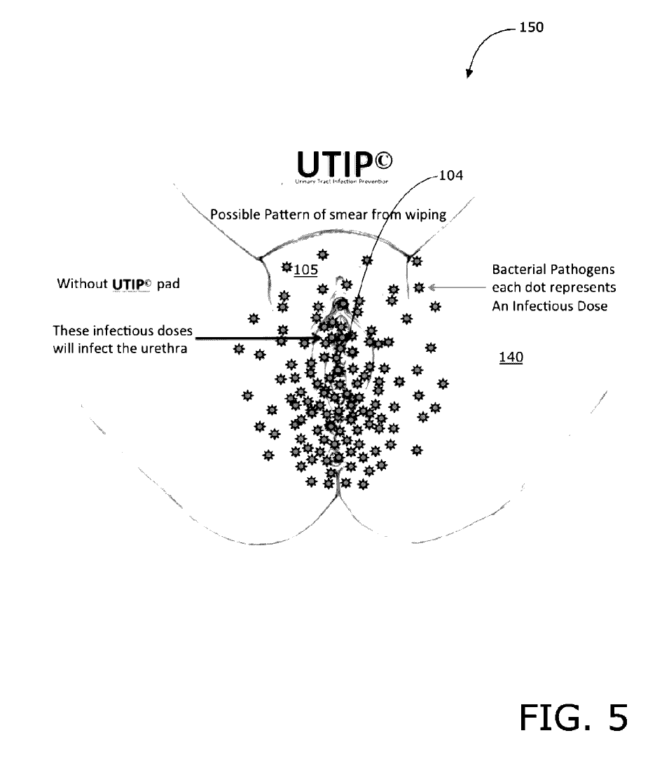

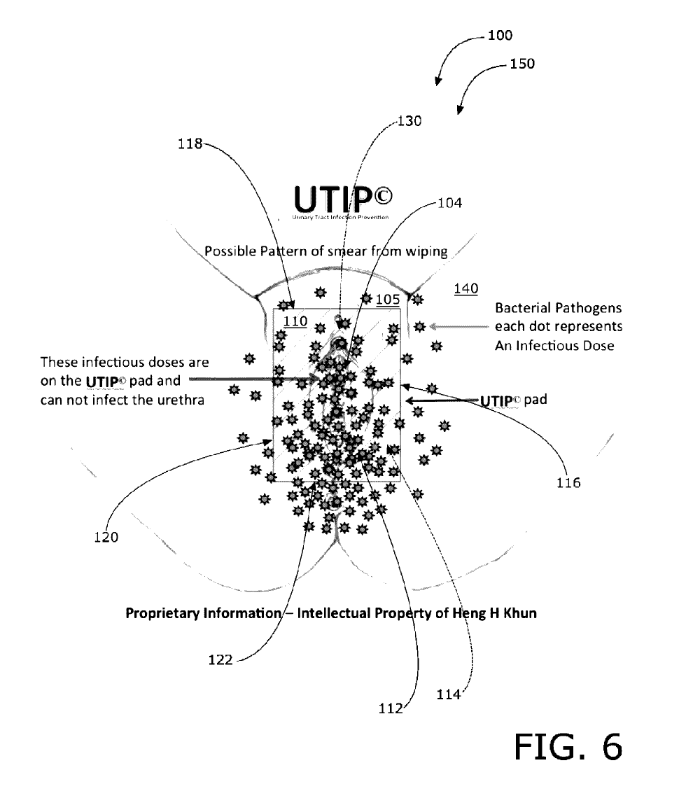

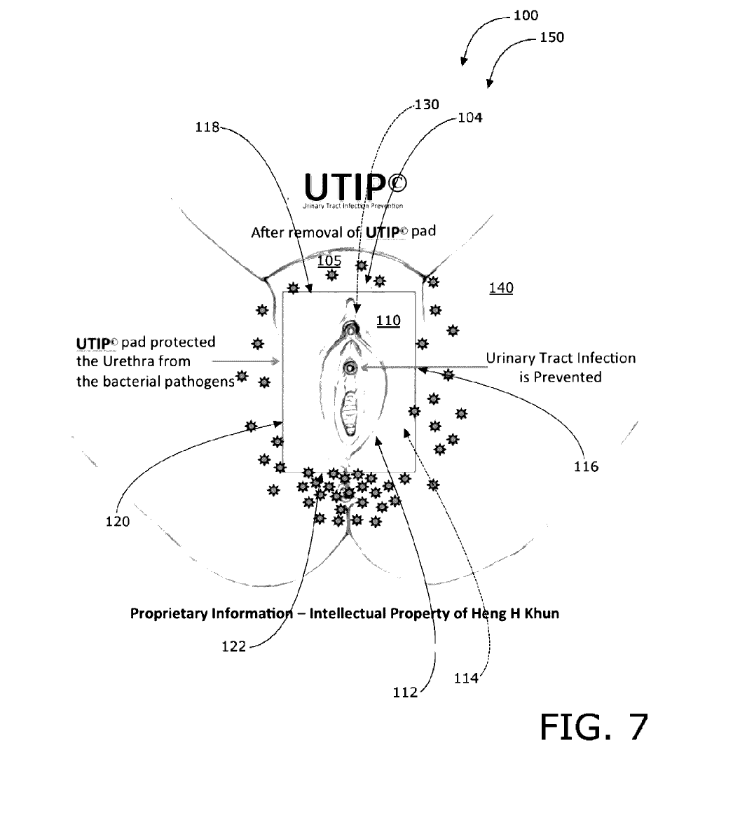

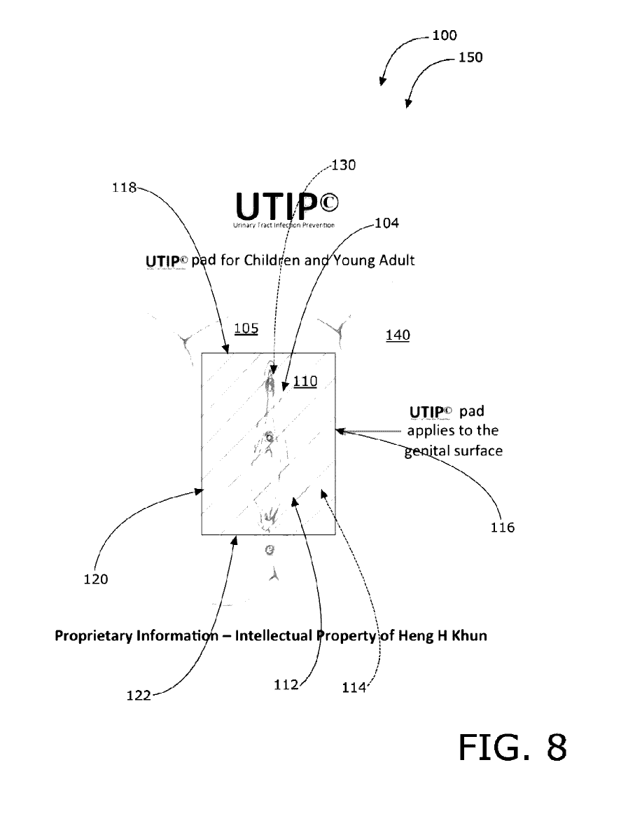

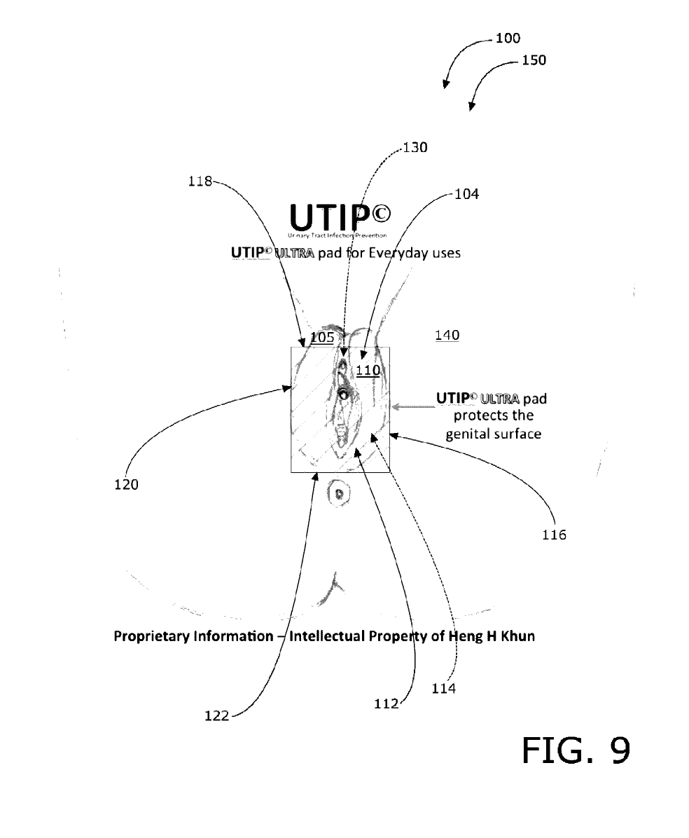

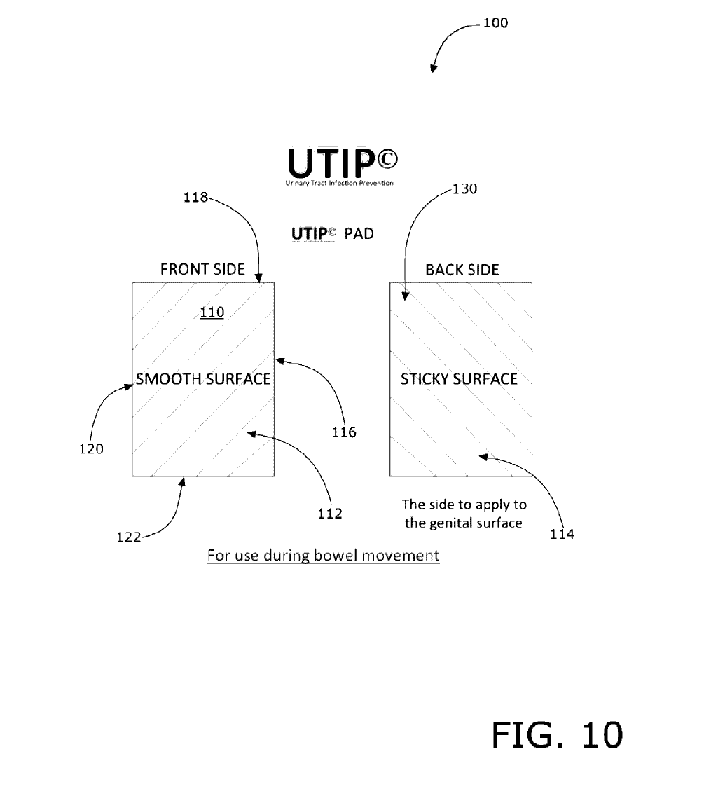

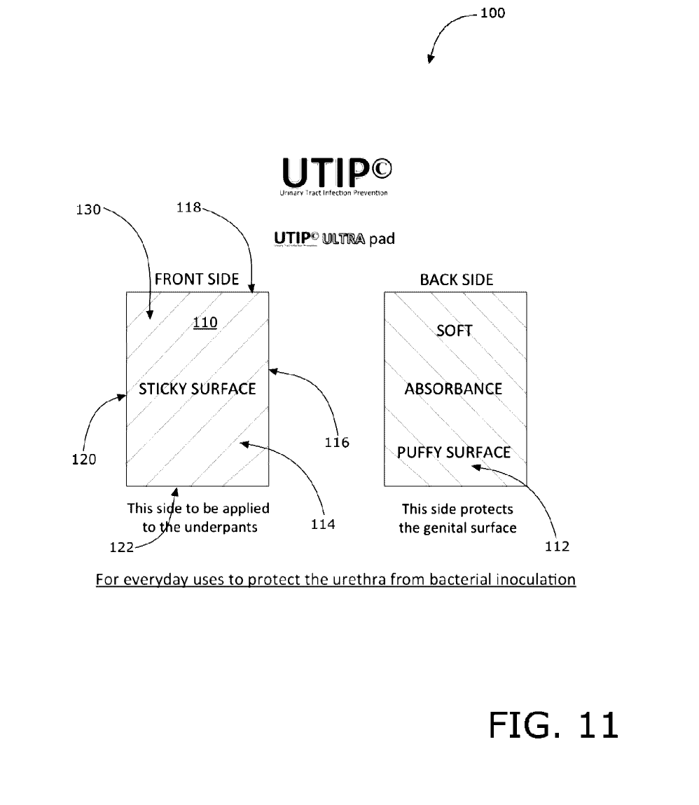

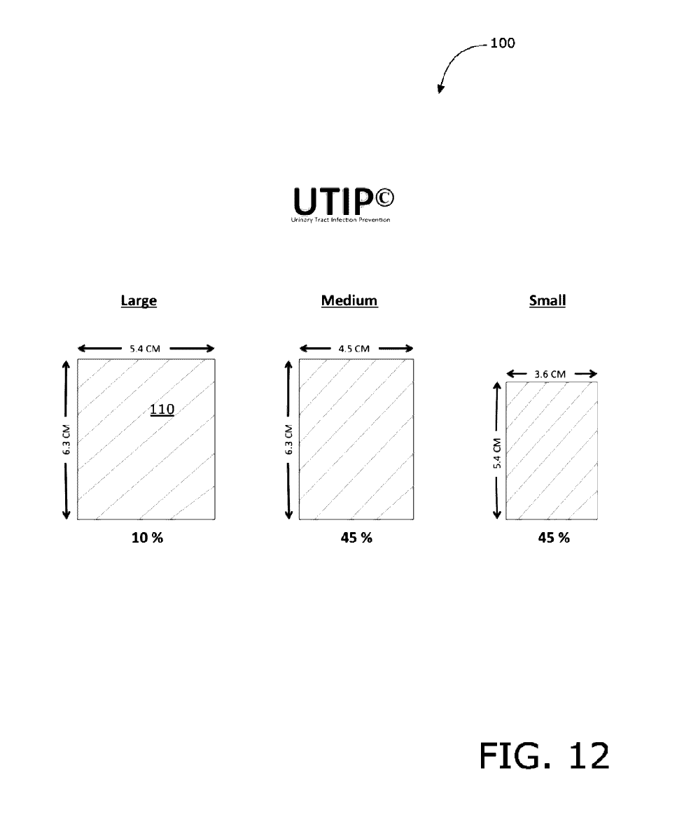

Referring now to the drawings more specifically by numerals of reference there is shown in FIGS. 1-12 various views of female urinary barrier system 100 designed for applying body 110 to pubic area 105 of vagina 104 of female-user 140.

Female urinary barrier system 100 may comprise body 110 and attacher 130. Body 110 may comprise top surface 112, bottom surface 114, first side 116, second side 118, third side 120, fourth side 122, and thickness 124. Parameters of body 110 of female urinary barrier system 100 may be defined by top surface 112, bottom surface 114, first side 116, second side 118, third side 120, fourth side 122, and thickness 124. Further, thickness 124 of body 110 of female urinary bather system 100 may comprise united layer 126. Top surface 112 of body 110 of female urinary barrier system 100 may comprise a material useful for preventing penetration of fecal matter and other contaminants that are harmful to a female urethra through body 110 of female urinary barrier system 100.

Female urinary barrier system 100 may comprise a rectangular profile. Alternatively, first side 116, second side 118, third side 120, and fourth side 122 of body 110 of female urinary barrier system 100 may comprise a curved profile. As should be appreciated, body 110 is disposable after use.

Body 110 may be adhesively attached to a pair of underwear 205 via attacher 130; attacher 130 of female urinary barrier system 100 may comprise adhesive 210. Bottom surface 114 of body 110 of female urinary bather system 100 may comprise adhesive 210 such that body 110 may be able to be applied to pubic area 105 of female-user 140 for purposes of prevention of cross-contamination from an anus of female-user 140 to vagina 104 of female-user 140 during a bowel movement cleaning.

In one embodiment of the present invention, attacher 130 may comprise adhesive 210 applied to entire area of bottom surface 114 of body 110 of female urinary barrier system 100 for purposes of creating seal around vaginal opening of pubic area 105 of female-user 140. Attacher may comprise bio-adhesive applied to outer periphery of bottom surface 114 of body 110 of female urinary barrier system 100 for purposes of creating seal around vaginal opening of female-user 140. Attacher 130 may comprise bio-adhesive, and alternatively bio-based pressure sensitive adhesives useful for attachment to skin of female-user 140 surrounding vagina 104 of female-user 140. Attacher 130 may comprise hand applicable pressure-sensitive hydrogels useful for attachment to skin of female-user 140 surrounding vagina 104 of female-user 140. Attacher 130 of bottom surface 114 of body 110 may comprise (adhesive glue and) range of 1-10 percent medical grade silicon.

Female urinary barrier system 100 comprising body 110 preferably has a rectangular profile. United layer 126 of thickness 124 of body 110 may comprise 0.1-10 percent cotton and paper useful for prevention of penetration of fecal contaminants through at least one united layer 126 of thickness 124 of body 110 of female urinary barrier system 100. Other percentages and combinations in materials may be used. Top of body 110 may comprise invasive infection prevention coating for purposes of prevention of penetration of contaminated liquids through at least one united layer 126 of thickness 124 of body 110 of female urinary barrier system 100.

United layer 126 of thickness 124 of body 110 of female urinary barrier system 100 may comprise Parylene coating barrier in order to provide ultra-thin anti-microbial pinhole free coating. Body 110, when properly applied by female-user 140, may be a bather that protects vagina 104 of female-user 140 from fecal matter and other harmful pathogenic bacteria, viruses, or fungi from cross-contamination of Genus's Enterococcus, Escherichia, Klebsiella, Pseudomonas, Candida, Chlamydia, and Mycoplasma. Body 110, when properly applied by female-user 140, may further be a barrier that protects (vaginal opening and) urethra of female-user 140 from fecal matter and other harmful pathogenic bacteria, viruses, or fungi from cross-contamination of Genus's Enterococcus, Escherichia, Klebsiella, Pseudomonas, Candida, Chlamydia, and Mycoplasma. Body 110, when properly applied by female-user 140, may prevent urinary tract infections in female-user 140 by use of adhesive pad placed over a genital surface before a bowel movement (or just prior to wiping).

In one embodiment, first side 116 and third side 120 may be manufactured with approximate dimensions of 6.3 centimeters in length and second side 118 and fourth side 122 may be manufactured with approximate width of 5.4 centimeters useful for a relatively large pubic area 105 of female-user 140. In another embodiment, first side 116 and third side 120 may be manufactured with approximate dimensions of 6.3 centimeters in length and second side 118 and fourth side 122 may be manufactured with approximate width of 4.5 centimeters useful for a relatively medium pubic area 105 of female-user 140. In another embodiment first side 116 and third side 120 may be manufactured with approximate dimensions of 5.4 centimeters in length and second side 118 and fourth side 122 may be manufactured with approximate width of 3.5 centimeters useful for a relatively small pubic area 105 of female-user 140. In this way the present invention is appropriately sized to fit a range of pubic areas/sizes and to give proper surface area coverage, as necessary.

A method of use for female urinary bather system 100 according to an embodiment of the present invention may comprise the steps of: step one, opening a sanitary package containing female urinary barrier system 100; removal of the adhesive covering from the pad; applying body 110 to vagina 104 of female-user 140 via attacher 130; depressing body 110; completion of the wiping process; removing body 110; and disposing of body 110.

It should be noted that the steps described in the method of use can be carried out in many different orders according to user preference. The use of “step of” should not be interpreted as “step for”, in the claims herein and is not intended to invoke the provisions of 35 U.S.C. §112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A female urinary barrier system comprising:
a body having;
a top surface;
a bottom surface;
a first side;
a second side;
a third side;
a fourth side; and
a thickness; and
an attacher;
wherein said female urinary barrier system comprises said body and said attacher;
wherein parameters of said body of said female urinary barrier system are defined by said top surface, said bottom surface, said first side, said second side, said third side, said fourth side, and said thickness;
wherein said thickness of said body of said female urinary barrier system comprises at least one united layer;
wherein said top surface of said body of said female urinary barrier system comprises a material useful for preventing the penetration through said body of said female urinary barrier system of fecal matter and other contaminants that are harmful to a female urethra and vaginal opening;
wherein said attacher of said female urinary barrier system comprises at least one adhesive; and
wherein said bottom surface of said body of said female urinary barrier system comprises said adhesive such that said body is able to be applied to a pubic area of a female-user for purposes of prevention of cross-contamination from said user's anus to said users vagina during a bowel movement cleaning.

2. The female urinary barrier system of claim 1 wherein said body comprises a rectangular profile.

3. The female urinary barrier system of claim 1 wherein said first side, said second side, said third side, and said fourth side of said body comprises a curved profile.

4. The female urinary barrier system of claim 1 wherein said body is disposable after use.

5. The female urinary barrier system of claim 1 wherein said at least one united layer of said thickness of said body comprises 0.1-10 percent cotton and paper useful for prevention of penetration of fecal contaminants through said united layers of said thickness of said body of said female urinary barrier system.

6. The female urinary barrier system of claim 1 wherein said top of said body comprises an invasive infection prevention coating for purposes of prevention of penetration of contaminated liquids through said united layers of said thickness of said body of said female urinary barrier system.

7. The female urinary barrier system of claim 1 wherein said attacher comprises an adhesive applied to an entire area of said bottom surface of said body of said female urinary barrier system for purposes of creating a seal around said female-user's vaginal opening of said female-user's pubic area.

8. The female urinary barrier system of claim 1 wherein said attacher comprises a bio-adhesive applied to an outer periphery of said bottom surface of said body of said female urinary barrier system for purposes of creating a seal around said female-user's vaginal opening.

9. The female urinary barrier system of claim 8 wherein said attacher comprises said bio-adhesive, and alternatively a bio-based pressure sensitive adhesives useful for attachment to said female-users skin surrounding a vagina of said female-user.

10. The female urinary barrier system of claim 9 wherein said attacher comprises hand applicable pressure-sensitive hydrogels useful for attachment to said female-users skin surrounding said vagina of said female-user.

11. The female urinary barrier system of claim 8 wherein said attacher of said bottom of said body of said female urinary barrier system comprises adhesive glue and a range of 1-10 percent medical grade silicon.

12. The female urinary barrier system of claim 1 wherein one of said united layers of said thickness of said body of said female urinary barrier system comprises a Parylene coating barrier in order to provide an ultra-thin anti-microbial pinhole free coating.

13. The female urinary barrier system of claim 1 wherein said body, when properly applied by said female-user, is a barrier that protects said vagina of said female-user from fecal matter and other harmful pathogenic bacteria, viruses, or fungi from cross-contamination of Genus's Enterococcus, Escherichia, Klebsiella, Pseudomonas, Candida, Chlamydia, and Mycoplasma.

14. The female urinary barrier system of claim 1 wherein said body, when properly applied by said female-user, is a barrier that protects a urethra of said female-user from fecal matter and other harmful pathogenic bacteria, viruses, or fungi from cross-contamination of Genus's Enterococcus, Escherichia, Klebsiella, Pseudomonas, Candida, Chlamydia, and Mycoplasma.

15. The female urinary barrier system of claim 1 wherein said body, when properly applied by said female-user, prevents urinary tract infections in said female-user(s) by use of an adhesive pad placed over a genital surface before a bowel movement.

16. The female urinary barrier system of claim 1 wherein said first side and said third side are manufactured with approximate dimensions of 6.3 centimeters in length and said second side and said fourth side are manufactured with approximate width of 5.4 centimeters useful for a relatively large female said pubic area.

17. The female urinary barrier system of claim 1 wherein said first side and said third side are manufactured with approximate dimensions of 6.3 centimeters in length and said second side and said fourth side are manufactured with approximate width of 4.5 centimeters useful for a relatively medium female said pubic area.

18. The female urinary barrier system of claim 1 wherein said first side and said third side are manufactured with approximate dimensions of 5.4 centimeters in length and said second side and said fourth side are manufactured with approximate width of 3.5 centimeters useful for a relatively small female said pubic area.

19. A female urinary barrier system comprising:
a body having;
a top surface;
a bottom surface;
a first side;
a second side;
a third side;
a fourth side; and
a thickness; and
an attacher;
wherein said female urinary barrier system comprises said body and said attacher;
wherein parameters of said body of said female urinary barrier system are defined by said top surface, said bottom surface, said first side, said second side, said third side, said fourth side, and said thickness;
wherein said thickness of said body of said female urinary barrier system comprises at least one united layer;
wherein said top surface of said body of said female urinary barrier system comprises a material useful for preventing penetration through said body of said female urinary barrier system of fecal matter and other contaminants that are harmful to a female urethra and vaginal opening;
wherein said attacher of said female urinary barrier system comprises at least one adhesive; and
wherein said bottom surface of said body of said female urinary barrier system comprises said adhesive such that said body is able to be applied to an undergarment of a female-user for purposes of prevention of bacterial inoculation from exterior sources.

20. A method of using a female urinary barrier system comprising the steps of:
opening;
applying;
depressing;
wiping;
removing; and
disposing.
